(19) United States
(12) Patent Application Publication
(10) Pub. No.: US 2023/0363945 A1
(43) Pub. Date: Nov. 16, 2023

(54) LASER PULSE CONTROL WITH SUB-CARRIER MODULATION

(71) Applicant: Alcon Inc., Fribourg (CH)

(72) Inventors: David Jung, Tustin, CA (US); Keith Watanabe, Irvine, CA (US)

(21) Appl. No.: 18/307,983

(22) Filed: Apr. 27, 2023

Related U.S. Application Data
(60) Provisional application No. 63/364,429, filed on May 10, 2022.

(51) Int. Cl. A61F 9/008; A61B 18/20

(52) U.S. Cl. CPC A61F 9/00823; A61B 18/20; A61F 2009/00891; A61F 2009/00887; A61F 2009/0087; A61B 2018/205547

(57) ABSTRACT

Systems and methods are disclosed for flexibly controlling laser pulses being output from a laser system. An example surgical system comprises a laser, a laser energy control system configured to regulate the amount of electromagnetic energy of each laser pulse that exits the laser system, and a laser pulse controller. The control signals communicated by the laser pulse controller may include a sub-carrier signal that modulates the amount of electromagnetic energy of the laser pulses that exit the laser system. The control signals may further include a threshold signal and/or a maximum power signal. The sub-carrier signal may oscillate between the threshold power and a maximum power.

### TECHNICAL FIELD

The present disclosure is directed to systems and methods for controlling laser pulses being output from a laser system.

### BACKGROUND

Lasers are used in many different medical procedures including a number of different ophthalmic procedures. For example, lasers may be used in cataract surgery, such as for fragmenting the cataractous lens. In some procedures, a laser is used for initial fragmentation of the lens, followed by phacoemulsification of the lens by an ultrasonic handpiece to complete the breakdown of the lens for removal. In other procedures, the laser may be used for complete fragmentation and/or phacoemulsification of the lens for removal, without the need for a separate application of ultrasonic energy. Lasers may also be used for other steps in cataract surgery, such as for making the corneal incision(s) and/or opening the capsule.

Lasers may also be used in glaucoma surgery. For example, a laser may be used to form all or part of a channel through the trabecular meshwork or scleral tissue for drainage of aqueous humor from the eye.

Lasers may also be used in vitreoretinal surgery. In some procedures, a laser may be used for vitrectomy, to sever or break the vitreous fibers for removal. The laser may be incorporated into a vitrectomy probe, and the energy from the laser may be applied to the vitreous fibers to sever or break the vitreous fibers for removal.

In other vitreoretinal applications, lasers may be used for photocoagulation of retinal tissue. Laser photocoagulation may be used to treat issues such as retinal tears and/or the effects of diabetic retinopathy.

U.S. Patent Application Publication No. 2018/0360657 discloses examples of an ophthalmic laser system. That application describes laser uses such as for forming surgical cuts or for photodisrupting ophthalmic tissue as well as for cataract surgery, such as laser-assisted cataract surgery (LACS). U.S. Patent Application Publication No. 2019/0201238 discloses other examples of an ophthalmic laser system. That application describes laser uses such as in a vitrectomy probe for severing or breaking vitreous fibers. U.S. Patent Application Publication No. 2018/0360657 and U.S. Patent Application Publication No. 2019/0201238 are expressly incorporated by reference herein in their entirety.

Some laser systems emit pulses, with the pulses having a desired duration and repetition rate. Operating a laser in pulses can achieve desirable power and energy characteristics for a particular application. In addition, while the energy of a beam emitted by a laser can be controlled by controlling the laser itself, in some systems it is desirable to control the amount of energy of a laser beam downstream from the laser. Existing systems for laser pulse selection typically have one or more drawbacks, such as power loss, complexity, cost, etc. There is a need for improved systems and methods for laser pulse control.

### SUMMARY

The present disclosure is directed to improved systems and methods for controlling laser pulses being output from a laser system.

In some embodiments, a surgical system comprises: a laser configured to emit electromagnetic radiation in laser pulses, a laser energy control system configured to regulate the amount of electromagnetic energy of each laser pulse that exits the laser system, and a laser pulse controller configured to communicate control signals to the laser energy control system. The control signals communicated by the laser pulse controller to the laser energy control system may include a sub-carrier signal that modulates the amount of electromagnetic energy of the laser pulses that exit the laser system.

The sub-carrier signal may be in a periodic pattern. The sub-carrier signal may be in a square wave pattern. The sub-carrier signal may be in a sinusoidal pattern.

The control signals communicated by the laser pulse controller to the laser energy control system may further include a threshold signal representing a threshold power and/or a maximum power signal representing a maximum power. The threshold power and/or the maximum power may be adjustable. The sub-carrier signal may oscillate between the threshold power and a maximum power.

In some examples, the surgical system may further comprise an adjustable input device configured to be actuated over an operating range. The operating range of the adjustable input device may be configured to allow an operator to control dynamically the amount of energy of the laser pulses emitted from the laser that is output from the laser system. The operating range of the adjustable input device may be configured to allow an operator to control dynamically the amount of energy of the laser pulses emitted from the laser that is output from the laser system up to the amount of the maximum power, or between the threshold power and the maximum power. The adjustable input device may comprise a foot pedal configured to be actuated over the operating range.

In some examples, the surgical system may further comprise an optical switching device configured to switch between a first condition in which it allows laser pulses emitted from the laser to be output from the laser system and a second condition in which it prevents laser pulses emitted from the laser from being output from the laser system. The optical switching device may comprise a shutter and a shutter motor.

In some examples, the laser energy control system may comprise: a waveplate, a waveplate motor, and a polarizer plate, wherein the waveplate motor is configured to move the waveplate into different positions corresponding to different percentages of laser electromagnetic energy permitted to pass through the laser energy control system.

In some examples, a method of controlling a surgical system comprises: (i) providing input to the surgical system, wherein the surgical system comprises a laser configured to emit electromagnetic radiation in laser pulses, a laser energy control system configured to regulate the amount of electromagnetic energy of each laser pulse that exits the laser system, and a laser pulse controller configured to communicate control signals to the laser energy control system, wherein the control signals communicated by the laser pulse controller to the laser energy control system include a sub-carrier signal that modulates the amount of electromagnetic energy of the laser pulses that exit the laser system; (ii) emitting electromagnetic radiation from a laser in laser pulses; and (iii) outputting laser pulses from the laser system in accordance with the control signals communicated by the laser pulse controller. The sub-carrier signal may be in a periodic pattern. The control signals may further include a threshold signal and/or a maximum power signal.

Further examples and features of embodiments of the invention will be evident from the drawings and detailed description.

### BRIEF DESCRIPTION OF THE DRAWINGS

The accompanying drawings illustrate example implementations of the systems and methods disclosed herein and, together with the description, serve to explain the principles of the present disclosure.

FIG. 1 shows an example ophthalmic surgical console with a foot pedal connected to it.

FIG. 2 shows an example of architecture for a surgical system comprising a laser system.

FIG. 3 shows an example of architecture for a laser pulse controller.

FIG. 4 shows an example operating range for an adjustable input device such as a foot pedal.

FIG. 5 shows an example packet of instructions for sending to a laser pulse controller.

FIG. 6A shows an example of laser pulses emitted from a laser.

FIG. 6B shows an example of a static pulse control signal.

FIG. 6C shows the output of laser pulses in accordance with the static pulse control signal of FIG. 6B.

FIG. 7A shows an example of laser pulses emitted from a laser, similar to FIG. 6A.

FIG. 7B shows an example of a pulse control signal in linear mode.

FIG. 7C shows the output of laser pulses in accordance with the linear mode pulse control signal of FIG. 7B.

FIG. 8A shows an example of laser pulses emitted from a laser, similar to FIGS. 6A and 7A.

FIG. 8B shows an example of a sub-carrier signal and threshold signal.

FIG. 8C shows an output of laser pulses in accordance with the sub-carrier signal and threshold signal of FIG. 8B.

FIG. 9A shows an example of laser pulses emitted from a laser, similar to FIGS. 6A, 7A, and 8A.

FIG. 9B shows another example of a sub-carrier signal and threshold signal.

FIG. 9C shows an output of laser pulses in accordance with the sub-carrier signal and threshold signal of FIG. 9B.

The accompanying drawings may be better understood by reference to the following detailed description.

### DETAILED DESCRIPTION

For the purposes of promoting an understanding of the principles of the disclosure, reference will now be made to the implementations illustrated in the drawings, and specific language will be used to describe those implementations and other implementations. It will nevertheless be understood that no limitation of the scope of the claims is intended by the examples shown in the drawings or described herein. Any alterations and further modifications to the illustrated or described systems, devices, instruments, or methods, and any further application of the principles of the present disclosure, are fully contemplated as would normally occur to one skilled in the art to which the disclosure relates. In particular, the features, components, and/or steps described with respect to one implementation of the disclosure may be combined with features, components, and/or steps described with respect to other implementations of the disclosure. For simplicity, in some instances the same reference numbers are used throughout the drawings to refer to the same or like parts.

The designations “first” and “second” as used herein are not meant to indicate or imply any particular positioning or other characteristic. Rather, when the designations “first” and “second” are used herein, they are used only to distinguish one component from another. The terms “attached,” “connected,” “coupled,” and the like mean attachment, connection, coupling, etc., of one part to another either directly or indirectly through one or more other parts, unless direct or indirect attachment, connection, coupling, etc., is specified.

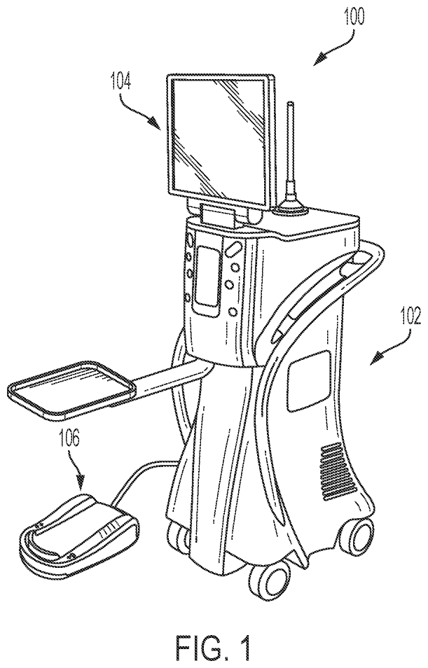

FIG. 1 shows an example ophthalmic surgical console 100 with a foot pedal 106 connected to it. The example ophthalmic surgical console 100 may be used in systems and methods in accordance with the present disclosure. The ophthalmic surgical console 100 may be similar to ophthalmic surgical consoles as shown and described in U.S. Pat. No. 9,931,447, the entire disclosure of which is hereby expressly incorporated herein by reference. The ophthalmic surgical console 100 may be similar to ophthalmic surgical consoles that have been known and used, such as the CENTURION® Vision System available from Alcon Laboratories, Inc. (Fort Worth, Texas) or the CONSTELLATION® Vision System available from Alcon Laboratories, Inc. (Fort Worth, Texas), or any other ophthalmic surgical console suitable for use with the principles described herein.

As shown in FIG. 1, the example ophthalmic surgical console 100 includes a housing 102 with a computer system disposed therein and an associated display screen 104 showing data relating to system operation and performance during an ophthalmic surgical procedure.

The foot pedal 106 is an adjustable input device that an operator may actuate over an operating range for controlling one or more functions. The foot pedal 106 may be pressed downward to various positions over the operating range to control functioning as described further below. While a foot pedal 106 is shown, other adjustable input devices, such as hand-operated buttons or knobs, may be used. The foot pedal 106 or other adjustable input device may be connected to the surgical console 100 by a wired or wireless connection.

The surgical console 100 includes one or more systems that may be used in performing an ophthalmic surgical procedure. For example, the surgical console 100 may include a fluidics system that includes an irrigation system for delivering fluid to the eye and an aspiration system for aspirating fluid from the eye.

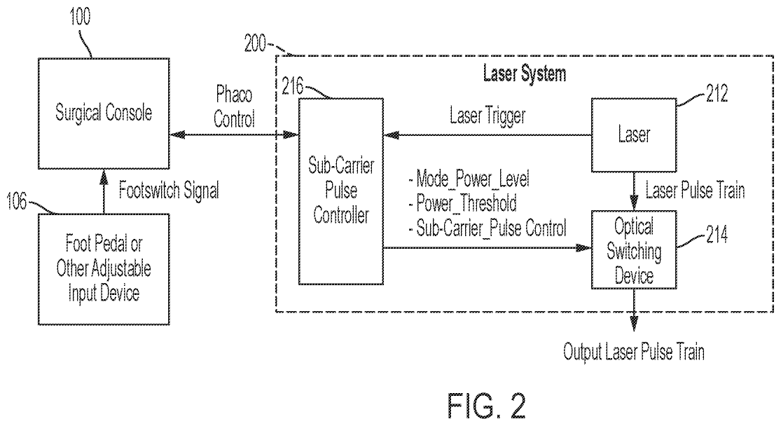

An example surgical system in accordance with this disclosure may include a laser system suitable for one or more ophthalmic procedures. FIG. 2 shows an example of architecture for a surgical system, including a surgical console 100, an adjustable input device, e.g., foot pedal 106, and an example laser system 200. The laser system 200 may comprise a laser 212, an optical switching device 214, and a laser pulse controller 216. In the illustrated embodiment, the laser pulse controller 216 is or includes a sub-carrier pulse controller. In some embodiments, the laser system 200 may be housed within the surgical console 100. In other embodiments, the laser system 200 may be housed in a separate console that communicates with the surgical console 100. In other embodiments, one or more parts of the laser system 200, such as the laser 212 and optical switching device 214, may be housed in a separate console that communicates with the surgical console 100, and one or more other parts of the laser system 200, such as the laser pulse controller 216, may be housed in the surgical console 100. In other embodiments, the laser system 200 may be in a stand-alone housing that receives input from a foot pedal or other adjustable input device 106 without the need for a separate surgical console 100.

In addition to the laser 212, optical switching device 214, and laser pulse controller 216, the laser system 200 may have other components. For example, the laser system 200 may include components for operating the laser, such as a power supply, laser pumps, laser energy control, and monitor. In addition, the laser system 200 may include other components in the optical path of the laser output, such as one or more lenses, mirrors, and optical fibers (not shown).

In some embodiments, the laser system 200 may be suitable for cataract surgery. In some embodiments, the output energy of the laser system is suitable for fragmentation and/or emulsification a cataractous lens. In some examples, the laser output is used for fragmentation and/or phacoemulsification of the lens to a sufficient degree for removal of the lens.

In some embodiments, the laser system 200 may be suitable for glaucoma surgery. In some embodiments, the output energy of the laser system is suitable for making or facilitating the formation of a drainage channel in eye tissue.

The laser 212 may be any type of laser suitable for the desired application. The laser 212 may output suitable electromagnetic radiation at any suitable wavelength. For example, the laser 212 may emit electromagnetic radiation in one or more wavelengths in the visible, infrared, and/or ultraviolet wavelengths. The laser 212 may operate or be operated to emit a continuous beam of electromagnetic radiation. Alternatively, the laser 212 may operate or be operated to emit a pulsed beam.

In one example, the laser 212 operates in the infrared range. For example, the laser 212 may output electromagnetic radiation in the mid-infrared range, for example in a range of about 2.0 microns to about 4.0 microns. Some examples wavelengths include about 2.5 microns to 3.5 microns, such as about 2.775 microns, about 2.8 microns, or about 3.0 microns. Such a laser may be suitable, for example, for lens fragmentation in cataract surgery, or for other procedures.

The laser system 200 is designed to direct the laser electromagnetic radiation from the laser 212 to an output port. The laser system 200 may direct the laser electromagnetic radiation from the laser 212 to the output port through one or more optical components, such as lenses and mirrors.

An instrument may be optically connected to the laser system 200 to receive the laser electromagnetic radiation from the output port. The instrument may be, for example, a handpiece for an ophthalmic procedure. The instrument or handpiece may be connected to the laser system by a delivery optical fiber. The delivery optical fiber may be flexible and relatively long to give the operator flexibility in maneuvering the handpiece at some distance away from the laser system 200. The laser electromagnetic radiation may be transmitted from the laser system 200, through the optical fiber and handpiece, and from an output tip of the handpiece to the desired target, such as a lens or lens fragment in the eye of a patient.

The optical switching device 214 is a device that operates either to allow laser electromagnetic radiation, e.g., laser pulses, emitted from the laser 212 to be output from the laser system or to prevent laser electromagnetic radiation, e.g., laser pulses, emitted from the laser 212 from being output from the laser system. The optical switching device 214 may switch back and forth between these two conditions, under the control of the laser pulse controller 216.

In some examples, the optical switching device 214 may comprise a shutter and a shutter motor. Examples of suitable optical switching devices are described and illustrated in U.S. Provisional Patent Application No. 63/186,387, the entirety of which is hereby incorporated by reference herein, and in U.S. Provisional Patent Application No. 63/222,521, the entirety of which is hereby incorporated by reference herein.

For example, the optical switching device 214 may comprise a shutter that is moved by the shutter motor into and out of the path of laser electromagnetic radiation, to selectively allow or prevent laser electromagnetic radiation from being output from the laser system. The shutter motor may be configured to move the shutter in an alternating manner between a first position corresponding to a first condition of the optical switching device (in which it allows laser electromagnetic energy, e.g., laser pulses, emitted from the laser to be output from the laser system) and a second position corresponding to a second condition of the optical switching device (in which it prevents laser electromagnetic energy, e.g., laser pulses, emitted from the laser from being output from the laser system). In an example, the shutter comprises a mirror, and the shutter motor comprises a galvanometer motor.

In another example, the optical switching device 214 may comprise: (i) a shutter having an axis of rotation and at least one open area and at least one solid area arranged around the axis of rotation of the shutter, and (ii) a shutter motor configured to rotate the shutter around the axis of rotation of the shutter. In such an example, the first condition of the optical switching device (in which it allows laser electromagnetic energy, e.g., laser pulses, emitted from the laser to be output from the laser system) corresponds to a position of the shutter in which a solid area of the shutter is not in a path of the laser pulses emitted from the laser, and the second condition of the optical switching device (in which it prevents laser electromagnetic energy, e.g., laser pulses, emitted from the laser from being output from the laser system) corresponds to a position of the shutter in which a solid area of the shutter is in the path of the laser pulses emitted from the laser.

The optical switching device 214 may further comprise a laser energy control system configured to regulate the amount of electromagnetic energy of each laser pulse that exits the laser system. For example, the laser energy control system may comprise a waveplate, a waveplate motor, and a polarizer plate, wherein the waveplate motor is configured to move the waveplate into different positions corresponding to different percentages of laser electromagnetic energy permitted to pass through the laser energy control system. Examples of such laser energy control systems are described and illustrated in U.S. Provisional Patent Application No. 63/186,387, the entirety of which is hereby incorporated by reference herein, and in U.S. Provisional Patent Application No. 63/222,521, which, as mentioned above, are both incorporated by reference herein.

In another alternative embodiment, the optical switching device 214 may comprise a pockels cell. A pockels cell optical switching device may switch back and forth, under the control of the laser pulse controller 216, between a first condition in which it allows laser pulses emitted from the laser to be output from the laser system and a second condition in which it prevents laser pulses emitted from the laser from being output from the laser system. Also, a pockels cell optical switching device can be operated incrementally to allow different percentages of electromagnetic energy emitted by the laser to be output by the laser system.

The laser pulse controller 216 is configured to communicate optical switching control signals to the optical switching device 214. The optical switching control signals are based on inputs to the surgical system, including from the adjustable input device, e.g., foot pedal 106, if provided.

The optical switching device 214 may comprises a power control device and a pulse picking device. The pulse picking device may comprise any suitable pulse picking device, including but not limited a shutter-based pulse picking device as described above. The power control device may comprise any suitable power control device, including but not limited to a waveplate-based power control device as described above. In alternative embodiments, a pockels cell arrangement may serve as the pulse picking device and/or the power control device. The laser system 200 may further comprise a beam polarizer. The laser pulse controller 216 sends laser power control signals and pulse picking control signals to the optical switching device 214. As described above, a handpiece may be connected, e.g., by a cable with an optical fiber, to an output port of the laser system 200. The output laser pulse train from the laser system 200 travels through the optical fiber and handpiece to the target (e.g., cataractous lens, trabecular meshwork, scleral tissue, other tissue, etc.).

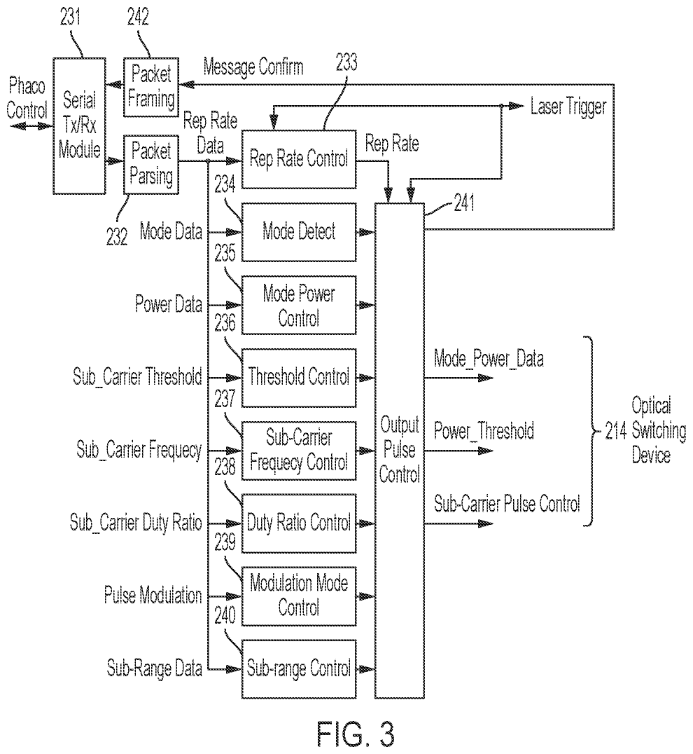

FIG. 3 shows an example of architecture for a laser pulse controller 216. As would be understood by persons having ordinary skill in the art, the use of controllers in processing environments may be implemented in software, firmware, hardware or some suitable combination of software, firmware, and/or hardware, such as software loaded into a processor and executed. The laser pulse controller 216 may be implemented in software, firmware, hardware or some suitable combination of software, firmware, and/or hardware, such as software loaded into a processor and executed.

The example laser pulse controller 216 comprises a serial transmitter/receiver (Tx/Rx) module 231 that communicates with a serial communication (Tx/Rx) controller or similar device (e.g., similar UART, CAN Bus, or Ethernet device) of the surgical console 100. In use, the surgical console 100 sends packets of data to the laser pulse controller 216, which are received by the serial Tx/Rx module 231. As described in more detail below, the packets may include data based, at least in part, on input from the adjustable input device 106. A packet parsing module 232 of the laser pulse controller 216 is configured to parse the packet data. In the illustrated example, the packet parsing module 232 sends repetition rate data to a repetition rate control module 233, mode data to a mode detect module 234, power data to a mode power control module 235, sub-carrier threshold data to a threshold control module 236, sub-carrier frequency data to a sub-carrier frequency control module 237, sub-carrier duty ratio data to a duty ratio control module 238, pulse modulation data to a modulation mode control module 239, and sub-range data to a sub-range control module 240. The repetition rate control module 233 also receives a laser trigger input signal, indicating the timing of the beginning of each laser pulse. The repetition rate control module 233 sends signals indicating the repetition rate of the laser to an output pulse control module 241, which may also receive a laser trigger input signal. The output pulse control module 241 also receives input signals from the mode detect module 234, mode power control module 235, threshold control module 236, sub-carrier frequency control module 237, duty ratio control module 238, modulation mode control module 239, and sub-range control module 240 based on their respective input data.

The output pulse control module 241 of the laser pulse controller 216 sends control signals to the optical switching device 214, wherein the control signals may be based, in part, on input from the adjustable input device 106. The control signals communicated by the laser pulse controller 216 to the optical switching device 214 may comprise a mode power signal (e.g., Mode_Power_Data), which controls the maximum laser pulse power output. The control signals communicated by the laser pulse controller 216 to the optical switching device 214 may also comprise a power threshold signal (e.g., Power_Threshold), which sets a threshold amount as described below. The control signals communicated by the laser pulse controller 216 to the optical switching device 214 may also comprise a sub-carrier pulse control signal (e.g., Sub-Carrier Pulse Control), which controls the sub-carrier signal as described below. A repetition rate signal may be sent to control the repetition rate of the laser pulses emitted by the laser.

As described in more detail below, the sub-carrier pulse control signal may be used for providing control of laser pulse output. In some examples, the sub-carrier pulse control signal may be used to establish a sub-carrier frequency or cycle. The sub-carrier pulse control signal may include a duty ratio, or a separate sub-carrier duty ratio signal may be provided, that establishes the ratio of the amount of the sub-carrier signal waveform above the central axis to the amount of the waveform below the central axis, as discussed further below.

The output pulse control module 241 of the laser pulse controller 216 may send message confirm signals to a packet framing module 242. The packet framing module 242 assembles the data from the message confirm signal and sends it as packets of data to the serial Tx/Rx module 231. The Tx/Rx module 231 then sends the packets of data based on the message confirm signals to the serial Tx/Rx controller of the surgical console 100 to confirm the signals from the laser pulse controller 216.

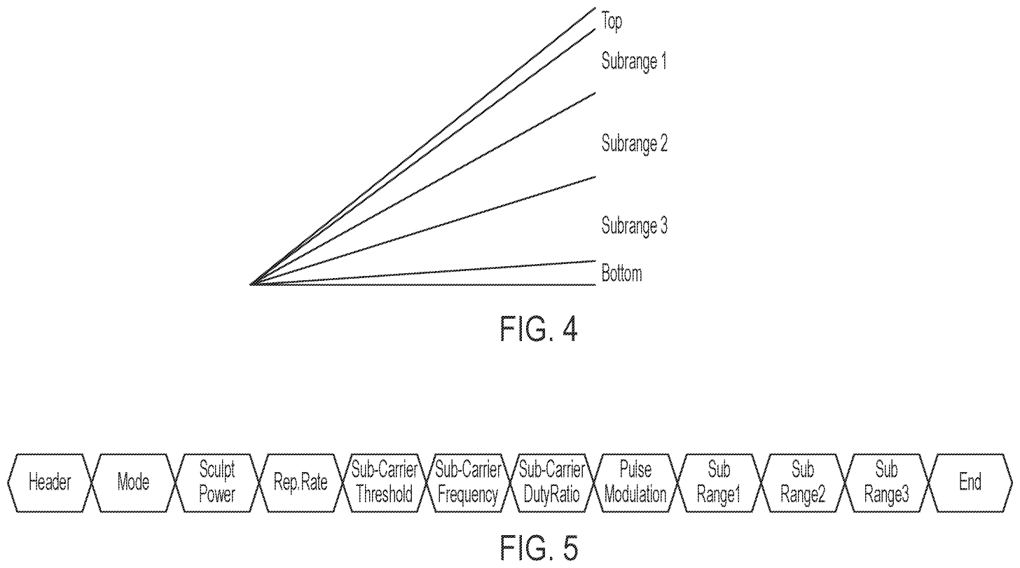

FIG. 4 shows an example operating range for an adjustable input device such as a foot pedal 106. The foot pedal 106 or other adjustable input device can be actuated by an operator over the operating range to control the laser output. In the example of a foot pedal, the operator depresses the foot pedal by the desired amount to move the foot pedal into the desired area of the operating range. In other examples, such as hand-operated buttons or knobs, the operator moves or tunes the input device into the desired area of the operating range. In some embodiments, the foot pedal or other adjustable input device may be adjustable in real time during a surgical procedure, giving the operator the ability to dynamically control the laser pulses being output from the laser system during a procedure.

Many examples of different functioning over the operating range are possible. In the illustrated example, the operating range includes three subranges, but more or fewer subranges may be used.

The following is a description of one of many examples. When the adjustable input device is moved or tuned to subrange 1, the surgical console may be activated for a specific function, such as irrigation, without any laser output. When the adjustable input device is moved or tuned to subrange 2, the surgical console may be activated for a different function, such as aspiration, without any laser output. The irrigation function may continue to operate in subrange 2. When the adjustable input device is moved or tuned to subrange 3, the laser system may be activated to output laser electromagnetic energy. The irrigation and/or aspiration functions may continue to operate in subrange 3. By moving or tuning the adjustable input device within subrange 3, the operator may dynamically adjust the laser output, as described below.

Many variations are possible. For example, subrange 2 and 3 in the above example may be reversed, such that laser control occurs in subrange 2 and aspiration occurs in subrange 3.

In one example, adjustment of the adjustable input device controls the percentage of electromagnetic energy of the laser pulses that are output. That is, the laser emits laser pulses at a specific energy, and the input from the adjustable input device is used to adjust the laser energy control system of the optical switching device 214 to control the percentage of energy of the laser pulses that are output from the laser system. Based on the input from the adjustable input device, the power level signal, which is sent by the laser pulse controller 216 to the optical switching device 214, may be adjusted to control the amount of energy of the laser pulses output from the laser system. For example, the top of subrange 3 may correspond to 0% of laser energy output, the bottom of subrange 3 may correspond to 100% of laser energy output, and positions in between may correspond to increments in the range of 0% to 100%. In other examples, the operating range of the adjustable input device is configured to allow an operator to control dynamically the percentage of laser pulses emitted from the laser that are output from the laser system. In other examples, adjusting the adjustable input device into subrange 3, or to a specific point in subrange 3, can act as an on-off switch that triggers operation of the laser system at the set output.

One or more inputs to the system, e.g., from a touchscreen (with graphical user interface), button, dial, knob, foot pedal, adjustable input device, or other input device, may be used to control the laser system to output only certain of the laser pulses emitted by the laser. That is, the laser emits laser pulses at a specific repetition rate, and the input is used to control the optical switching device 214 to switch back and forth between the first condition in which it allows laser pulses emitted from the laser to be output from the laser system and the second condition in which it prevents laser pulses emitted from the laser from being output from the laser system. One or more of the inputs to the system may comprise or be part of the console 100, the adjustable input device 106, and/or an external control system (e.g., with its own touchscreen (with graphical user interface), button, dial, knob, or other input device).

In certain embodiments, a user input controls a sub-carrier frequency, which controls the length of a sub-carrier cycle, and a duty ratio. Based on the input, the laser pulse controller sends signals (e.g., Sub-Carrier Pulse Control signals) to the optical switching device and controls the sub-carrier frequency and duty ratio. For example, if the repetition rate of the laser is 1000 Hz, a sub-carrier frequency of 100 Hz results in 10 pulses per cycle. By selecting input that controls the duty ratio, a range of different pulses per cycle may be output (e.g., a range from 1 to 9, from 1 to 10, from 0 to 9, from 0 to 10, etc.), thereby controlling the percentage of laser pulses that are output. As another example, if the repetition rate of the laser is 1000 Hz, a sub-carrier frequency of 10 Hz results in 100 pulses per cycle. By selecting input that controls the duty ratio, a range of different pulses per cycle may be output (e.g., a range from 1 to 99, from 1 to 100, from 0 to 99, from 0 to 100, etc.), thereby controlling the percentage of laser pulses that are output.

In some examples, the repetition rate of the laser and the energy output of the laser, including different energy outputs of the laser, if desired, may also be selected by the adjustable input device or another input device, such as a touchscreen, button, dial, knob, or other input.

FIG. 5 shows an example packet of instructions for sending to a laser pulse controller. The packet includes the following data: Header, Mode, Sculpt Power (or Mode Power), Repetition Rate, Sub-Carrier Threshold, Sub-Carrier Frequency, Sub-Carrier Duty Ratio, Pulse Modulation, Subrange 1, Subrange 2, Subrange 3, and End. The Header identifies the beginning of the packet. The Mode identifies which operating mode has been selected. The Sculpt Power (or Mode Power) identifies the selected power output of the laser; this may be a maximum power signal representing a maximum power output. The Repetition Rate identifies the rate of pulses to be emitted from the laser. The Sub-Carrier Threshold identifies the threshold level or threshold power, as discussed further below. The Sub-Carrier Frequency identifies the frequency of the sub-carrier signal. The Sub-Carrier Duty Ratio identifies the duty ratio of the sub-carrier signal. The Pulse Modulation identifies the type of pulse modulation, e.g., static or linear, with or without a sub-carrier signal. Subrange 1, Subrange 2, and Subrange 3 identify the position to which the adjustable input device has been moved or tuned, including the incremental position within the range (e.g., 0 to 100).

FIG. 6A shows an example of laser pulses emitted from a laser, each upward arrow representing a laser pulse. This shows the repetition rate of the laser pulses being emitted by the laser, which in this example is 1 KHz.

FIG. 6B shows an example of a static pulse control signal. The power level signal is set at 100%. In static mode, as shown, this power level is constant. In linear or variable mode, this power level is adjustable, e.g., by the adjustable input device (e.g., foot pedal).

FIG. 6C shows the output of laser pulses in accordance with the static pulse control signal of FIG. 6B. As can be seen, all laser pulses are output, at 100% power.

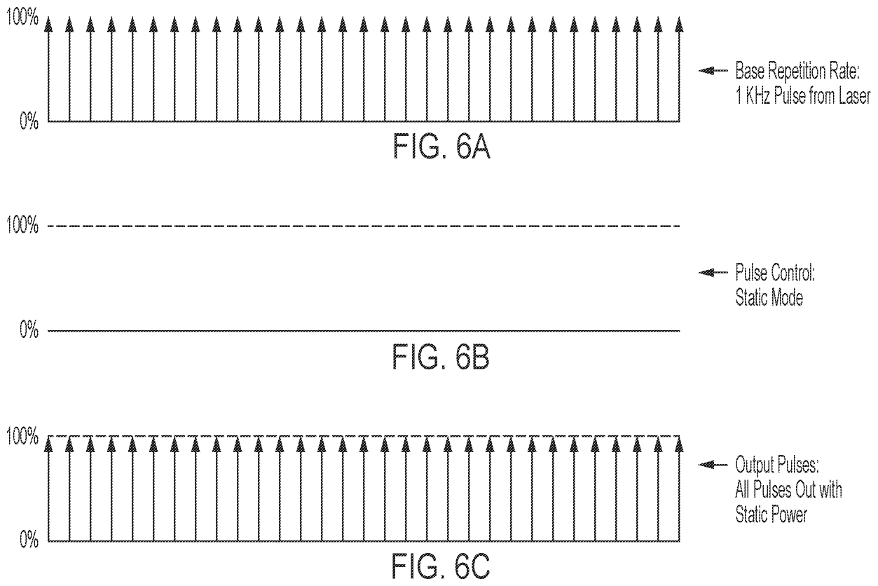

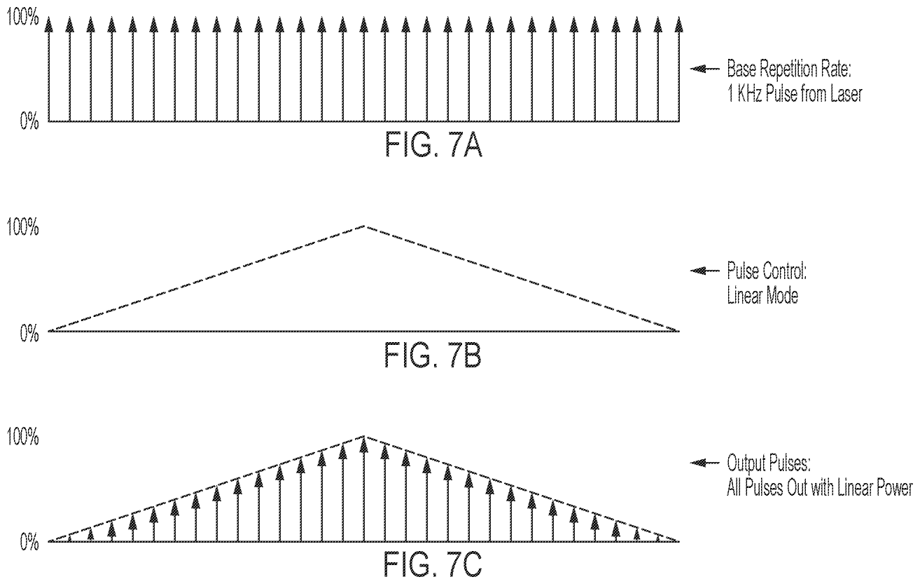

FIG. 7A shows an example of laser pulses emitted from a laser, similar to FIG. 6A. Like FIG. 6A, this shows the repetition rate of the laser pulses being emitted by the laser, which in this example is 1 KHz.

FIG. 7B shows an example of a linear pulse control signal. The power level signal is adjusted over time. In linear (or variable) mode, as shown, the power level is adjustable, e.g., by the adjustable input device (e.g., foot pedal).

FIG. 7C shows the output of laser pulses in accordance with the linear pulse control signal of FIG. 7B. As can be seen, all laser pulses are output, with different levels of power in accordance with the power control signal.

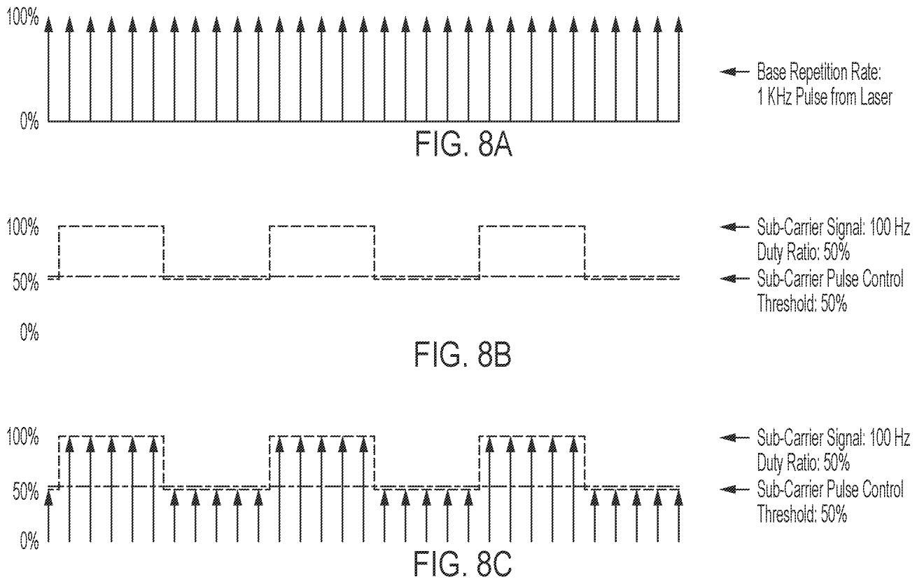

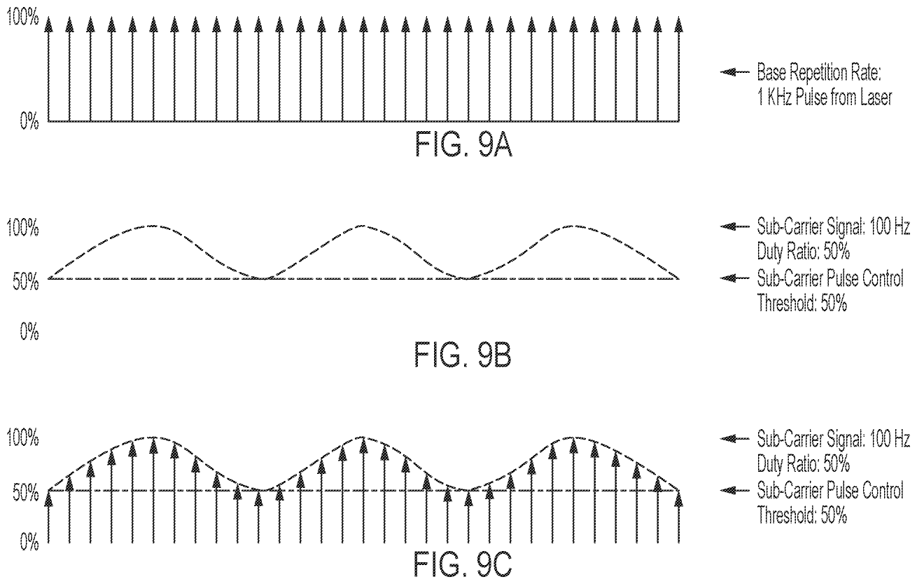

The operating modes in FIGS. 6A-6C and 7A-7C are similar in output to operating modes described and illustrated in U.S. Provisional Patent Application No. 63/256,071, the entirety of which is hereby incorporated by reference herein. For example, FIGS. 6C and 7C show outputs similar to sculpt mode described and illustrated in that application. FIGS. 8A-8C and 9A-9C illustrate how embodiments herein allow additional control over laser pulse output by use of a sub-carrier signal (sub-carrier frequency and duty ratio) and threshold signal.

FIG. 8A shows an example of laser pulses emitted from a laser, similar to FIGS. 6A and 7A. Like FIGS. 6A and 7A, this shows the repetition rate of the laser pulses being emitted by the laser, which in this example is 1 KHz.

FIG. 8B shows an example of a sub-carrier signal, shown as a waveform in a dashed line. The sub-carrier frequency in this example is 100 Hz, which with a 1 KHz repetition rate results in 10 laser pulses per sub-carrier cycle.

In the illustrated example, the sub-carrier duty ratio is set at 50% (or 50:50). The duty ratio establishes the ratio of the amount of the sub-carrier signal waveform above the central axis to the amount of the waveform below the central axis. That is, the waveform of the subcarrier signal has a central axis. In the illustrated example, the central axis corresponds to a level of 75% power. As can be seen, the sub-carrier oscillates in a waveform above and below the central axis. In the illustrated example, with a duty ratio of 50%, 50% of the sub-carrier signal waveform is above the central axis, while 50% of the sub-carrier signal waveform is below the central axis.

Other duty ratios are possible. For example, at a duty ratio of 30% (or 30:70), 30% of the sub-carrier signal waveform is above the central axis, while 70% of the sub-carrier signal waveform is below the central axis. At a duty ratio of 60% (or 60:40), 60% of the sub-carrier signal waveform is above the central axis, while 40% of the sub-carrier signal waveform is below the central axis. At a duty ratio of 95% (or 95:5), 95% of the sub-carrier signal waveform is above the central axis, while 5% of the sub-carrier signal waveform is below the central axis. Duty ratios are possible anywhere in the range of 0% to 100%, inclusive.

FIG. 8B also shows the sub-carrier threshold, shown as a horizontal dashed line. In this example, the threshold signal is at 50%, as shown. Threshold signals are possible anywhere in the range of 0% to 100%, inclusive.

The threshold signal identifies or represents the threshold level or threshold power. In the illustrated example, the threshold signal sets the bottom or minimum of the sub-carrier signal. In the illustrated example, the maximum power is set at 100%. As described further below, the maximum power may be fixed or adjustable. The sub-carrier signal oscillates between the threshold power, 50% in the illustrated example, and the maximum power, 100% in the illustrated example.

As described further below, the sub-carrier signal modulates the amount of electromagnetic energy of the laser pulses that exit the laser system. In some embodiments, like that illustrated in FIG. 8B, the sub-carrier signal is in a periodic pattern that is repeated. For example, the sub-carrier signal may oscillate between the threshold power and the maximum power, as shown in FIG. 8B. In some examples, like that illustrated in FIG. 8B, the sub-carrier signal may be in a square wave pattern. In other examples, the sub-carrier signal may be in a sinusoidal pattern. Other patterns for the sub-carrier signal may be used.

FIG. 8C shows the output of laser pulses in accordance with the pulse control signals of FIG. 8B. As can be seen, the sub-carrier signal modulates the amount of electromagnetic energy of the laser pulses that exit the laser system. That is, the amount of energy of each laser pulse that is output from the laser system is regulated by the sub-carrier signal. In the illustrated example, the laser pulses are modulated in a periodic pattern between 50% power and 100% power.

In some examples, all of the laser pulses may be output from the laser system, in accordance with the power indicated by the sub-carrier signal. In other examples, the system may operate such that when the sub-carrier signal indicates a power level below the threshold, the optical switching device is in a condition in which it prevents laser pulses emitted from the laser from being output from the laser system. That is, the optical switching device is configured to switch between a first condition in which it allows laser pulses emitted from the laser to be output from the laser system (e.g., when the sub-carrier signal indicates a power level above the threshold) and a second condition in which it prevents laser pulses emitted from the laser from being output from the laser system (e.g., when the sub-carrier signal indicates a power level below the threshold).

In some examples, e.g., static mode, an input (actuator, button, knob, touchscreen, foot pedal, etc.) may be used as an on-off switch that triggers operation of the laser system at a set output. For example, moving the input to the on position initiates operation of the laser system at the output levels shown in FIG. 8C. The input may be an adjustable input device such as a foot pedal. For example, adjusting the adjustable input device into subrange 3, or to a specific point in subrange 3, can act as an on-off switch that triggers operation of the laser system at the set output.

In some examples, e.g., linear or variable mode, adjustment of an adjustable input device may be used to control the percentage of electromagnetic energy of the laser pulses that are output. That is, the laser emits laser pulses at a specific energy, and the input from the adjustable input device is used to adjust the laser energy control system of the optical switching device 214 to control the percentage of energy of the laser pulses that are output from the laser system. For example, the top of subrange 3 may correspond to 0% of laser energy output, the bottom of subrange 3 may correspond to 100% of laser energy output, and positions in between may correspond to increments in the range of 0% to 100%. When used in conjunction with a sub-carrier signal such as that shown in FIGS. 8B and 8C, the maximum output is regulated by the sub-carrier signal. That is, adjusting the adjustable input device to 100% results in laser pulses up to the sub-carrier signal as shown in FIG. 8C, while lower percentages result in correspondingly lower amounts of power.

Thus, in such examples, the adjustable input device (e.g., foot pedal) is configured to be actuated over an operating range. The operating range of the adjustable input device is configured to allow an operator to control dynamically the amount of energy of the laser pulses emitted from the laser that is output from the laser system. In some examples, the operating range of the adjustable input device is configured to allow an operator to control dynamically the amount of energy of the laser pulses emitted from the laser that is output from the laser system up to the amount of the maximum power. In some examples, the operating range of the adjustable input device is configured to allow an operator to control dynamically the amount of energy of the laser pulses emitted from the laser that is output from the laser system between the threshold power and the maximum power.

Referring again to FIG. 8C, the output results in periods of higher power alternating with periods of lower power. When the power is in or transitioning to lower power, the output energy of the laser pulses is minimized. Certain procedures, e.g., irrigation and/or aspiration of tissue, may be timed to coincide with these periods. When the power is in or transitioning to higher power, the output energy of the laser pulses is maximized. Certain procedures, e.g., break-up of hard tissue, may be timed to coincide with these periods.

FIG. 9A shows an example of laser pulses emitted from a laser, similar to FIGS. 6A, 7A, and 8A. Like FIGS. 6A, 7A, and 8A, this shows the repetition rate of the laser pulses being emitted by the laser, which in this example is 1 KHz.

FIG. 9B shows another example of a sub-carrier signal, shown as a waveform in a dashed line. The sub-carrier frequency in this example is 100 Hz, which with a 1 KHz repetition rate results in 10 laser pulses per sub-carrier cycle.

In the illustrated example, the sub-carrier duty ratio is set at 50% (or 50:50). As discussed above, the duty ratio establishes the ratio of the amount of the sub-carrier signal waveform above the central axis to the amount of the waveform below the central axis. As discussed above, other duty ratios are possible, anywhere in the range of 0% to 100%, inclusive.

FIG. 9B also shows the sub-carrier threshold, shown as a horizontal dashed line. In this example, the threshold signal is at 50%, as shown. As discussed above, threshold signals are possible anywhere in the range of 0% to 100%, inclusive. The sub-carrier signal oscillates between the threshold power, 50% in the illustrated example, and the maximum power, 100% in the illustrated example. In the example of FIG. 9B, the sub-carrier signal may be in a sinusoidal pattern. Many other forms for the sub-carrier signal are possible (e.g, saw tooth, stair-step, etc.).

FIG. 9C shows the output of laser pulses in accordance with the pulse control signals of FIG. 9B. As can be seen, the sub-carrier signal modulates the amount of electromagnetic energy of the laser pulses that exit the laser system. That is, the amount of energy of each laser pulse that is output from the laser system is regulated by the sub-carrier signal. In the illustrated example, the laser pulses are modulated in a periodic pattern between 50% power and 100% power.

As discussed above, in some examples, all of the laser pulses may be output from the laser system, in accordance with the power indicated by the sub-carrier signal. In other examples, the system may operate such that when the sub-carrier signal indicates a power level below the threshold, the optical switching device is in a condition in which it prevents laser pulses emitted from the laser from being output from the laser system.

In some examples, as discussed above, an arrangement such as that shown in FIG. 9C may be operated in static mode, wherein an input may be used as an on-off switch that triggers operation of the laser system at a set output. In some examples, as discussed above, an arrangement such as that some in FIG. 9C may be operated in linear or variable mode, wherein adjustment of an adjustable input device may be used to control the percentage of electromagnetic energy of the laser pulses that are output.

An example method of controlling a surgical system as described herein is as follows. An operator selects inputs for the operating mode (e.g., static or linear, with or without sub-carrier), maximum power, repetition rate of the laser, sub-carrier frequency and/or duty ratio. In some embodiments, certain options may be provided for selection, wherein dependent on the selection by the operator, the surgical system sets the operating mode, maximum power, repetition rate of the laser, sub-carrier frequency and/or duty ratio. Alternatively, any of these parameters may be preset. The operator operates the system, with the laser output of a handpiece directed at the desired location (e.g., a cataractous lens, trabecular meshwork, scleral tissue, other tissue, etc.). Based on the input(s), and optionally other parameters, control signals are sent (e.g., by a packet as in FIG. 5) to a laser pulse controller. Based on the input, the laser pulse controller sends control signals to the optical switching device to control the laser output. The laser emits electromagnetic radiation from a laser in laser pulses. Based on the input(s), the optical switching device selectively controls the energy output of the laser pulses and/or selectively allows certain laser pulses to be output and prevents certain laser pulses from being output. In some embodiments, the operator may actuate the adjustable input device (e.g., foot pedal) over an operating range to control dynamically the power of the laser pulses being output from the laser system.

The operator may dynamically adjust the adjustable input device in real time to adjust the power output, i.e., the power level signal may be based on dynamic input from the adjustable input device. The operator may dynamically adjust the adjustable input device in real time to adjust the power level signal and, consequently, the amount of energy of the laser pulses to be output from the laser system.

The operator may switch inputs. The selected inputs may be based on the type of procedure, the stage of the procedure, the conditions, or other factors.

The ability to selectively output laser pulses and/or to control the laser output energy is useful for procedures in which laser control is advantageous. For example, in cataract surgery, it may be desirable to operate the laser system with high power for initially breaking up the lens. It may be desirable to operate the laser system with lower power for breaking up smaller pieces, so a lower energy level may be preferred. Pulse energy level control of laser pulses allows for a correct level of force to be applied to smaller particles which might otherwise be pushed away before they can be aspirated out of the eye by the irrigation system of the hand piece. As another example, for glaucoma treatment, it may be desirable to operate the laser system with a single laser pulse or just a few laser pulses for formation of a channel through eye tissue. It may also be desirable to use soft or low energy for certain glaucoma procedures.

As would be understood by persons of ordinary skill in the art, systems and methods as disclosed herein have advantages over prior systems and methods. For example, systems and methods as described herein allow simple, flexible, and/or dynamic control of laser pulses and/or energy, improving the ease, time, efficiency, accuracy, outcome, and/or cost of the procedures.

Persons of ordinary skill in the art will appreciate that the embodiments encompassed by the disclosure are not limited to the particular example embodiments described above. While illustrative embodiments have been shown and described, a wide range of modification, change, and substitution is contemplated in the foregoing disclosure. It is understood that such variations may be made to the foregoing without departing from the scope of the disclosure. Accordingly, it is appropriate that the appended claims be construed broadly and in a manner consistent with the disclosure.

What is claimed is:

1. A surgical system comprising:
a laser configured to emit electromagnetic radiation in laser pulses;
a laser energy control system configured to regulate the amount of electromagnetic energy of each laser pulse that exits the laser system; and
a laser pulse controller configured to communicate control signals to the laser energy control system;
wherein the control signals communicated by the laser pulse controller to the laser energy control system include a sub-carrier signal that modulates the amount of electromagnetic energy of the laser pulses that exit the laser system.

2. The surgical system as recited in claim 1, wherein the sub-carrier signal is in a periodic pattern.

3. The surgical system as recited in claim 2, wherein the sub-carrier signal is in a square wave pattern.

4. The surgical system as recited in claim 2, wherein the sub-carrier signal is in a sinusoidal pattern.

5. The surgical system as recited in claim 1, wherein the control signals communicated by the laser pulse controller to the laser energy control system further include a threshold signal representing a threshold power.

6. The surgical system as recited in claim 5, wherein the control signals communicated by the laser pulse controller to the laser energy control system further include a maximum power signal representing a maximum power.

7. The surgical system as recited in claim 6, wherein the maximum power is adjustable.

8. The surgical system as recited in claim 6, wherein the sub-carrier signal oscillates between the threshold power and a maximum power.

9. The surgical system as recited in claim 1, further comprising an adjustable input device configured to be actuated over an operating range.

10. The surgical system as recited in claim 9, wherein the operating range of the adjustable input device is configured to allow an operator to control dynamically the amount of energy of the laser pulses emitted from the laser that is output from the laser system.

11. The surgical system as recited in claim 9, wherein the control signals communicated by the laser pulse controller to the laser energy control system further include a maximum power signal representing a maximum power, and wherein the operating range of the adjustable input device is configured to allow an operator to control dynamically the amount of energy of the laser pulses emitted from the laser that is output from the laser system up to the amount of the maximum power.

12. The surgical system as recited in claim 9, wherein the control signals communicated by the laser pulse controller to the laser energy control system further include a threshold signal representing a threshold power and a maximum power signal representing a maximum power, and wherein the operating range of the adjustable input device is configured to allow an operator to control dynamically the amount of energy of the laser pulses emitted from the laser that is output from the laser system between the threshold power and the maximum power.

13. The surgical system as recited in claim 9, wherein the adjustable input device comprises a foot pedal configured to be actuated over the operating range.

14. The surgical system as recited in claim 1, further comprising an optical switching device configured to switch between a first condition in which it allows laser pulses emitted from the laser to be output from the laser system and a second condition in which it prevents laser pulses emitted from the laser from being output from the laser system.

15. The surgical system as recited in claim 13, wherein the optical switching device comprises a shutter and a shutter motor.

16. The surgical system as recited in claim 1, wherein the laser energy control system comprises:
a waveplate;
a waveplate motor; and
a polarizer plate;
wherein the waveplate motor is configured to move the waveplate into different positions corresponding to different percentages of laser electromagnetic energy permitted to pass through the laser energy control system.

17. A method of controlling a surgical system comprising:
(i) providing input to the surgical system, wherein the surgical system comprises:
a laser configured to emit electromagnetic radiation in laser pulses;
a laser energy control system configured to regulate the amount of electromagnetic energy of each laser pulse that exits the laser system; and
a laser pulse controller configured to communicate control signals to the laser energy control system;
wherein the control signals communicated by the laser pulse controller to the laser energy control system include a sub-carrier signal that modulates the amount of electromagnetic energy of the laser pulses that exit the laser system;
(ii) emitting electromagnetic radiation from a laser in laser pulses; and
(iii) outputting laser pulses from the laser system in accordance with the control signals communicated by the laser pulse controller.

18. The method of controlling a surgical system as recited in claim 17, wherein the sub-carrier signal is in a periodic pattern.

19. The method of controlling a surgical system as recited in claim 17, wherein the control signals communicated by the laser pulse controller to the laser energy control system further include a threshold signal representing a threshold power.

20. The method of controlling a surgical system as recited in claim 17, wherein the control signals communicated by the laser pulse controller to the laser energy control system further include a maximum power signal representing a maximum power, and the sub-carrier signal oscillates between the threshold power and a maximum power.
